(19) United States
(12) Patent Application Publication
(10) Pub. No.: US 2024/0206899 A1
(43) Pub. Date: Jun. 27, 2024

(54) HYGIENE DEVICE AND METHODS OF USE

(71) Applicant: Steven Mark Moss, Toronto (CA)

(72) Inventor: Steven Mark Moss, Toronto (CA)

(21) Appl. No.: 18/087,176

(22) Filed: Dec. 22, 2022

(51) Int. Cl. A61B 17/24

(52) U.S. Cl. CPC A61B 17/24; A61B 2017/246; A61M 31/00

(57) ABSTRACT

A hygiene device for use with a body orifice of a living being, the hygiene device including a head adapted for use in the body orifice, the head being formed of one or more absorbent materials that are adapted to collect substances from the body orifice and a handle connected to the head to enable the head to be inserted into and removed from the body orifice with the collected substances. Method of using the hygiene device are also provided.

### FIELD

This invention relates generally to hygiene devices, and more particularly to hygiene devices adapted for use with body orifices, such as a nose, of a living body and methods of use.

### BACKGROUND

The following background discussion is not an admission that anything discussed below is citable as prior art or common general knowledge. The documents listed below are incorporated herein in their entirety by this reference to them.

Hospitalizations and deaths from viruses such as influenza, rhinovirus and coronavirus are a serious problem for health care systems around the world. These viruses often enter the body through the nose (originating typically at the anterior nares). It is desirable to find ways to reduce the likelihood of such viruses, and other substances that may have a negative impact on health and wellness, from passing from the nose into the remainder of the body.

Similar problems exist for other body orifices where substances that may have a negative impact on health and wellness enter the body or collect before passing to the remaining portions of the body. Such substances may include viruses as well as fungi, biological organisms, bacteria, allergens, mucus or dirt.

There is a need for a device that is specifically adapted for collecting and removing at least some substances that may have a negative impact on health and wellness from a body orifice of a living being.

There is also a need for a device that is suitable for use on a regular basis by individuals that are not necessarily medically trained to collect and remove at least some substances from a body orifice. Such a device is preferably produced at a relatively low cost where all or a portion of such device is disposable after a single use.

There is also a need for a method of using a device on a regular basis by individuals that are not necessarily medically trained for collecting and removing at least some substances from a body orifice to reduce the risk of such substances entering the remainder of the living body.

### SUMMARY

In one aspect the invention provides a hygiene device for use with a body orifice of a living being, the hygiene device comprising:
a head adapted for use in the body orifice, said head being formed of one or more absorbent materials that are adapted to collect substances from the body orifice; and
a handle connected to said head to enable said head to be inserted into the body orifice to collect substances and to be subsequently removed from the body orifice to remove the collected substances.

In another aspect, the invention provides a method of using a device in a body orifice of a living body to collect and remove substances from the body orifice where such device includes a head formed of one or more absorbent materials that are adapted to collect substances from the body orifice and a handle connected to the head for inserting and removing the head from the body orifice, said method including the steps of:
selecting a device;
inserting the head of the device into the body orifice to collect substances from the body orifice;
removing the head of device with the collected substances from the body orifice.

In another aspect, the invention provides a use of a device in a body orifice of a living body to collect and remove substances where such device includes a head adapted for use in the body orifice, said head being formed of one or more absorbent materials that are adapted to collect substances from the body orifice; and a handle connected to said head for inserting and removing said head from the body orifice.

Other aspects and features of the teachings disclosed herein will become apparent, to those ordinarily skilled in the art, upon review of the following description of the specific examples of the specification.

### DRAWINGS

The drawings included herewith are for illustrating various examples of articles, methods, and apparatuses of the present specification and are not intended to limit the scope of what is taught in any way. For simplicity and clarity of illustration, where considered appropriate, reference numerals may be repeated among the drawings to indicate corresponding or analogous elements.

FIG. 1 is a schematic view of device in accordance with the present disclosure being used with a body orifice of a living being (in this instance a nose);

FIG. 2 is an exploded view of the device of FIG. 1;

FIG. 3 is a plan view of the device of FIG. 1;

FIG. 4 is a side view of the device of FIG. 1;

FIG. 5 is a first end view of the device of FIG. 1 showing the head;

FIG. 6 is a second end view of the device of FIG. 1 showing the handle, neck and head;

FIG. 7 is an enlarged view of the head of the device show in circle 7 of FIG. 3 showing a plurality of pores that are adapted for collecting or absorbing substances;

FIGS. 8(a) to 8(d) are plan views of different textured elements for the head of the device;

FIG. 9 is a cross-sectional view of the head for an alternative embodiment of the device having multiple layers of material;

FIG. 10 is a perspective view of an alternative embodiment of the device featuring asymmetric volumetric heads at both ends;

FIG. 11 is a plan view of the device of FIG. 10;

FIG. 12 is a side view of the device of FIG. 10;

FIG. 13 is an opposing side view of the device of FIG. 10;

FIG. 14 is an end view of the device of FIG. 10;

FIG. 15 is a perspective view of an alternate embodiment of the device having a head that may be releasably attached to a handle;

FIG. 16 is perspective view of the embodiment shown in FIG. 14 with the handle attached to the head;

FIGS. 17(a) and 17(b) are perspective views of a container containing a plurality of heads with a handle shown in FIG. 17(a) detached from head and in FIG. 17(b) attached to head;

FIG. 18 is a schematic view of an alternate embodiment of the device featuring a dispensing system for dispensing a desired solution to the head;

FIG. 19 is a schematic view of an alternate embodiment of the device featuring a different embodiment of dispensing system for dispensing a desired solution to the head; and

FIG. 20 is a flow chart showing a method of using the device in accordance with the present disclosure.

### DESCRIPTION OF VARIOUS EMBODIMENTS

Various apparatuses or methods will be described below to provide examples of the claimed invention. The claimed invention is not limited to apparatuses or methods having all of the features of any one apparatus or method described below or to features common to multiple or all of the apparatuses described below. The claimed invention may reside in a combination or sub-combination of the apparatus elements or method steps described below. It is possible that an apparatus or method described below is not an example of the claimed invention. The applicant(s), inventor(s) and/or owner(s) reserve all rights in any invention disclosed in an apparatus or method described below that is not claimed in this document and do not abandon, disclaim or dedicate to the public any such invention by its disclosure in this document.

A hygiene device in accordance with an embodiment of the present disclosure is shown generally at 10 in the figures. Hygiene device 10 includes a head 12 and a handle 14 as described in more detail below.

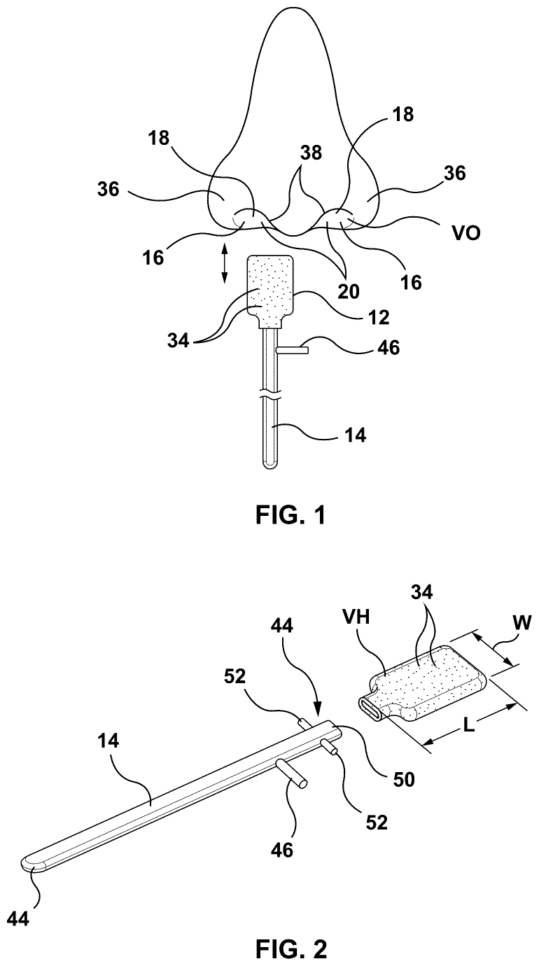

Hygiene device 10 is intended for use in a body orifice 16 of a living being (which may be a human or animal) to collect and remove at least some substances 18 from the body orifice 16 that may have a negative impact on the health and/or wellness of the living being if such substances 18 remain in place or pass, in whole or part, from orifice 16 into the remainder of the body. Body orifice 16 includes surrounding walls 20 of body that define body orifice 16. In a preferred embodiment, as shown in FIG. 1, body orifice 16 may be the nose and, more preferably, the anterior nares. Other body orifices 16 that may benefit from the use of device 10 include the mouth, ears, anus, urethra and vagina. The structure, shape and size of device 10 may differ depending on the intended body orifice 16.

Substances 18 may include viruses, such as influenza, coronavirus or rhinovirus, as well as fungi, biological organisms, bacteria, allergens, mucus, dirt or other substances that could be harmful to the health and/or wellness of the living being if allowed to remain in place or pass, in whole or part, to the remainder of the body.

Head 12 is formed of one or more materials 21 that are adapted to collect and remove substances 18 from body orifice 16 including its surrounding walls 20. Preferably, head 12 does not include flocked or bristled elements that may damage, irritate or tickle the orifice 16 or walls 20. Preferred materials 21 for head 12 include one or more medically safe absorbent materials such as an open cell material, more preferably an open cell foam material, and even more preferably an open cell polyurethane foam material. The preferred one or more materials 21 for head 12 have a plurality of pores 34 that are adapted for collecting one or more substances 18 for removal from orifice 16. Preferably, the one or more materials for head 12 have at least 10 pores per inch (ppi), more preferably at least 50 ppi, even more preferably between 50-500 ppi and most preferably between 80-120 ppi. Head 12 may be formed of a single layer of material or multiple layers as described further below.

Head 12 is shaped and sized to fit within the desired body orifice 16. In other words, head 12 may have different shapes and sizes according to the size and type of body orifice 16 where it is intended to be used. Head 12 may also be provided in adult and child sizes for the intended body orifice 16. Preferably, head 12 is sized to enter the body externally to the desired orifice 16. Preferably, head 12 has a volume VH that fills a significant portion of the volume VO of desired body orifice 16 (preferably at least 25% of the volume, more preferably at least 33% of the volume, even more preferably at least 50% of the volume, even more preferably at least 67% of the volume and most preferably at least 75% of the volume). This sizing provides a greater surface area and thus allows head 12 to collect and remove a greater amount of substances 18 from body orifice 16 and surrounding walls 20.

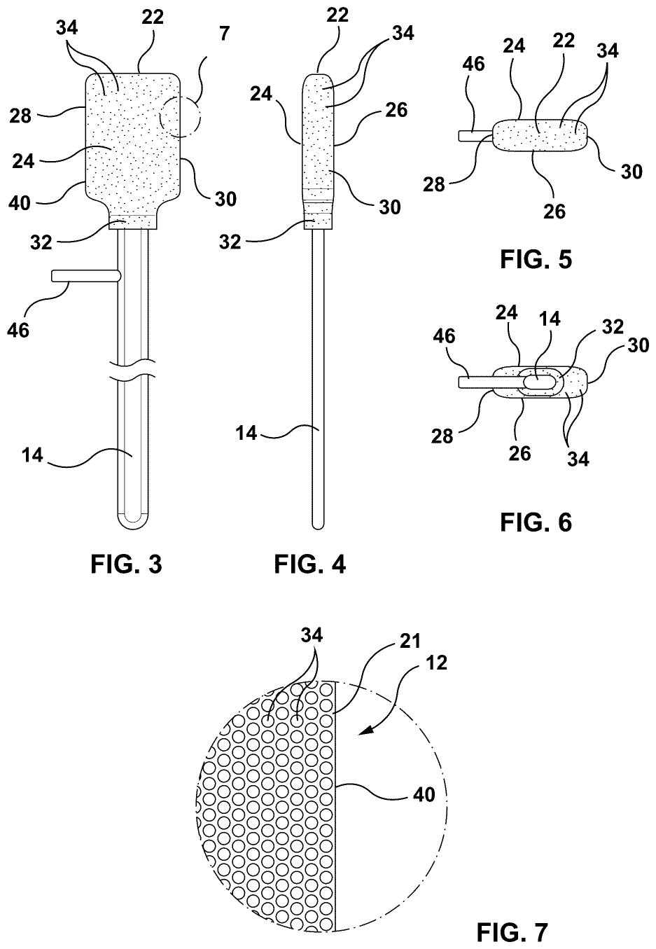

In one embodiment, as shown in FIGS. 1-7, head 12 includes an end 22, first and second opposing faces 24 and 26, first and second opposing sides 28 and 30 and a neck 32.

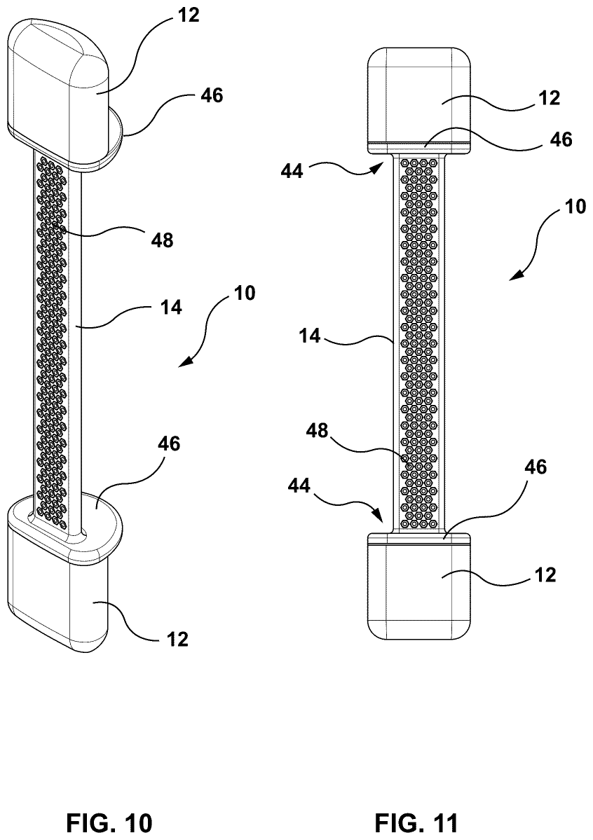

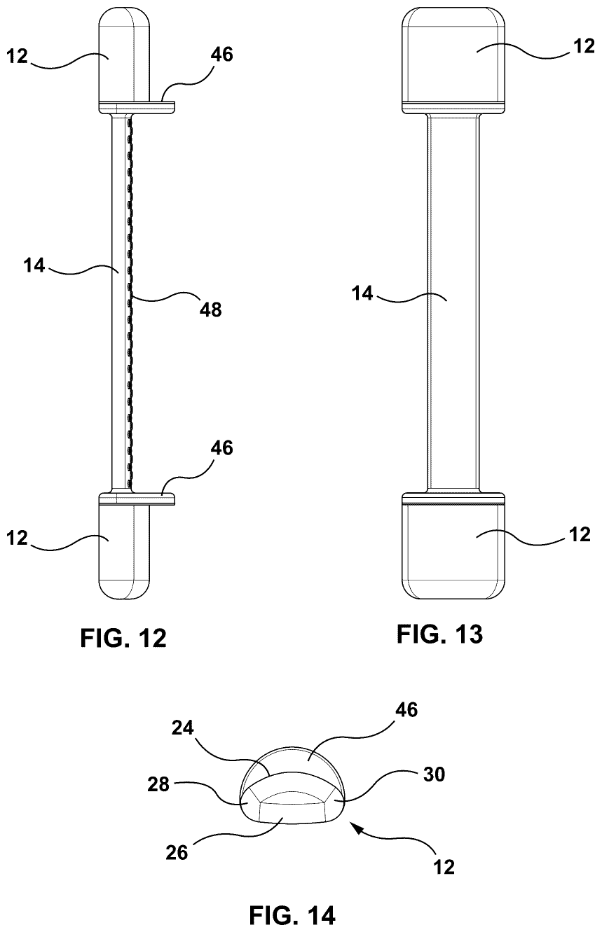

Head 12 may have a uniform symmetric volumetric shape as shown in FIGS. 1-7 or it may have an asymmetric volumetric shape as shown in FIGS. 10-14. For example, head 12 may have an asymmetric shape such as shown with the two headed version in FIGS. 10-14 that corresponds generally to the anatomic volumetric shape of the anterior nares. In this embodiment, opposing face 24 has a generally curved or convex shape adapted to match the generally curved or concave shape of the outer side 36, or alae, of the anterior nares while opposing face 26 has a generally flat shape adapted to match the generally flat shape of the inner side 38, or septum, of the anterior nares. End 22 and sides 28 and 30 are all preferably rounded as well to generally match the shape of the anterior nares and avoid any sharp edges.

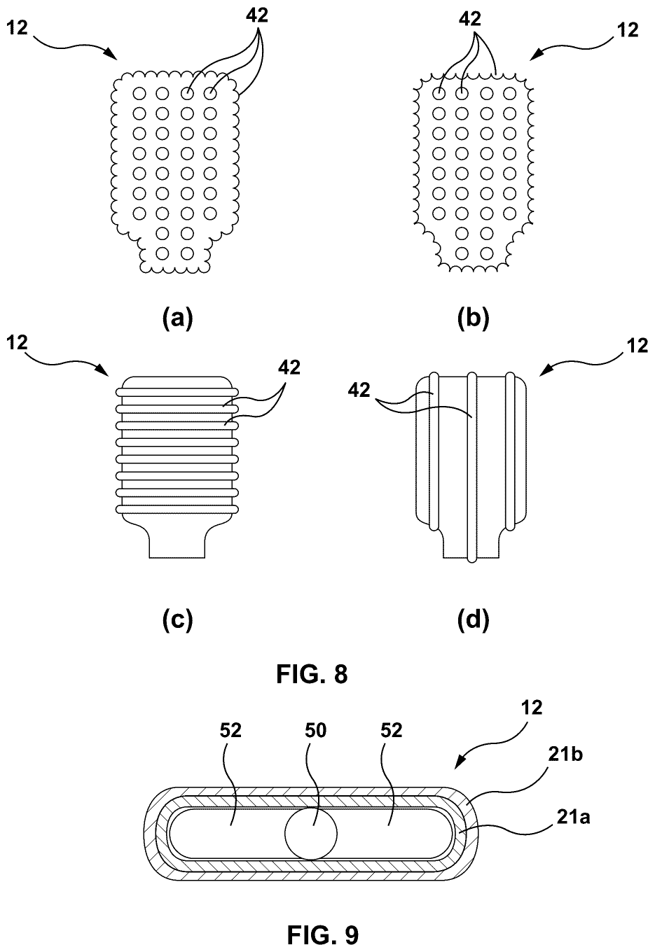

Head 12 has an outer surface 40 that may be substantially smooth and flat as shown in FIGS. 1-7 and FIGS. 10-14 or it may be textured to enhance the ability for head to collect and remove substances 18. Outer surface 40 may for example include textured elements 42 such as protrusions, indentations, ridges or swirls as shown in FIGS. 8(a) to 8(d). Preferably such textured elements are formed by molding of the material for head 12.

Head 12 may be formed from multiple layers of material 21a and 21b as shown in FIG. 9. Multiple layers of material may include an inner layer 21a and an outer layer 21b. Inner layer of material 21a may have different properties from outer layer of material 21b. For example, inner layer 21a may have a higher rate of absorbency than outer layer 21b. Outer layer 21b may also, or alternatively, be made of a softer material than inner layer 21a to reduce the risk of irritating the body orifice 16 including its surrounding walls 20.

Handle 14 is adapted to support head 12 and provide a grip to allow a user to insert head 12 into body orifice 16, to manipulate head 12 to collect and remove substances 18 and to remove head 12 with collected substances from body orifice 16. In one embodiment, as shown in FIGS. 1-7 head 12 is disposed on one of the ends 44 of handle 14. Handle may be solid (as shown) or hollow (as described further below).

In another embodiment, as shown in FIGS. 10-14, head 12 is disposed on both ends 44 of handle 14. The two-headed embodiment is advantageous in instances where there are two portions to body orifice 16 (such as two nostrils) so that the user may use a different head 12 for collecting and removing substances 18 from each portion. The two-headed embodiment is also advantageous in instances where each of heads 12 have a different shape, texture or function.

Handle 14 may include a flange 46 that is adapted to limit the extent or distance to which head 12 may be inserted into body orifice 16. Flange 46 may be adapted to engage an external portion of body orifice 16 to deter device 10 from being inserted a further distance into body orifice 16 or to at least provide a sensory cue to user that device 10 has been inserted a sufficient distance into body orifice 16. Handle may further include a textured surface 48 (as shown in FIGS. 10 and 11) to aid in gripping of handle 14.

Handle 14 may include a post 50 with a collar 52 at one or both ends 44 that is adapted to receive and support head 12 so that head 12 does not separate from handle 14 during use.

In one embodiment, a disposable single use device 10 may be provided with head 12 permanently attached to handle 14 by molding or securing head 12 onto post 50 over collar 52 with a mechanical, thermal or chemical bond. Preferably, head 12 and handle 14 are formed of biodegradable materials that minimize the impact on the environment once the device is disposed. Handle 14 may for example be formed from wood, bamboo, paper or from a biodegradable plastic. Head 12 may for example be formed from a paper material or from a biodegradable open cell foam.

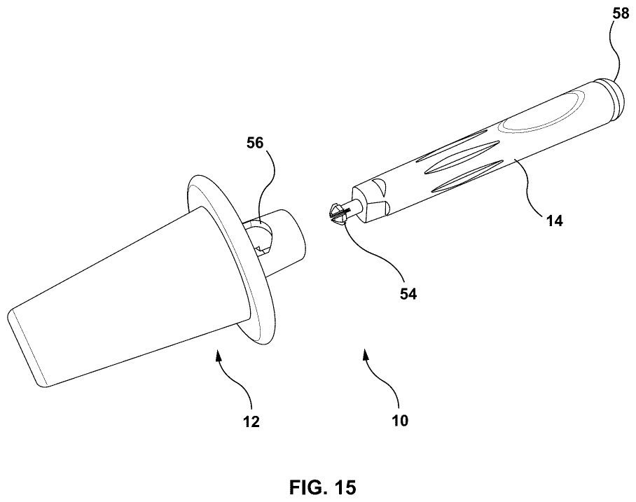

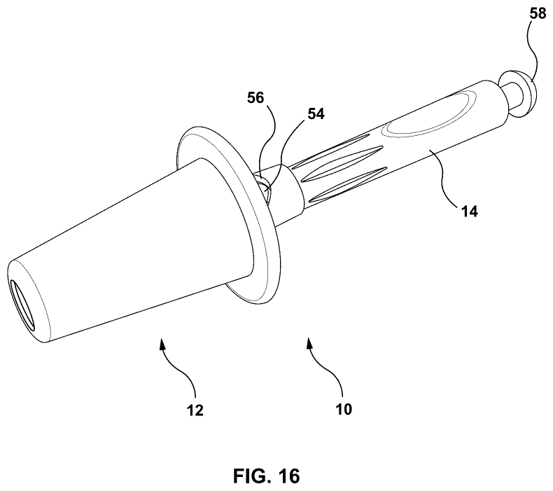

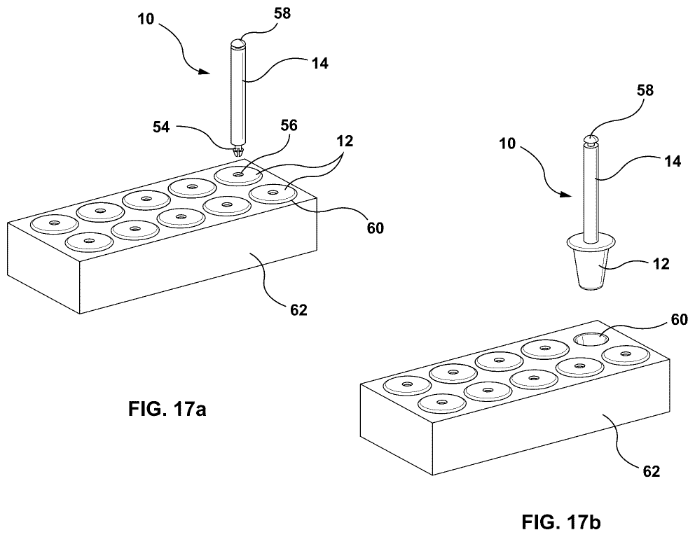

In an alternative embodiment, as shown in FIGS. 15 and 16, head 12 may be releasably attached to handle 14. Handle 14 may include a spring biased latch 54 that fits within a corresponding opening 56 defined within head 12. Latch 54 may be engaged and released by control 58. This allows a releasable connection for handle 14 to engage and release head 12. With this embodiment, a user may purchase a plurality of disposable single use replacement heads 12 that may for example be disposed in individual compartments 60 within a container 62 for use with the same handle 14 as shown in FIGS. 17(a) and 17(b). This has the advantage of reducing costs and disposable waste.

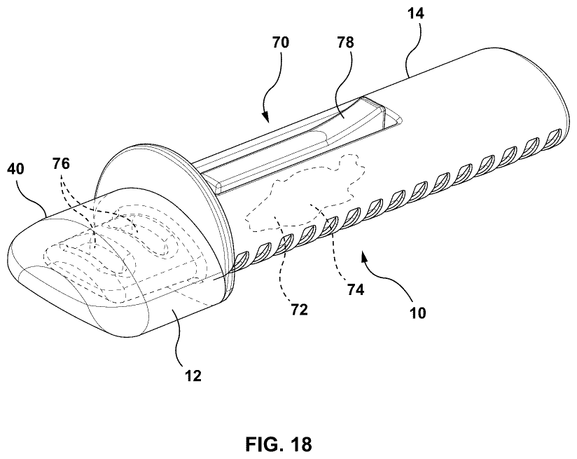

Referring to FIG. 18, an alternate embodiment of the device is shown with a dispensing system 70 for dispensing a desired solution 72 to head 12 in order for solution 72 to be distributed to body orifice 16.

Solution 72 may comprise a saline solution (preferably a 0.9% salt solution) or variants thereof (such as a ringer's lactate or sodium lactate solution). Solution 72 may also or alternatively comprise one or more of water, other salt solutions, minerals or metals (e.g., zinc), herbals, antibiotics, anti-inflammator anesthetics, preservatives, gels, aqua, hydroxypropyl methylcellulose (e.g., 1%), succinic acid (e.g., 1%), disodium succinate (e.g., 0.44%), PCA (e.g., 0.35%), phenethyl alcohol, zinc EDTA, zinc acetate, carrageenan or other desired ingredients.

Dispensing system 70 may include a reservoir 74 for containing a desired volume of solution 72 and one or more channels 76 for transferring solution 72 from reservoir 74 to head 12. Solution 72 preferably permeates the material of head 12 to outer surface 40. Reservoir 74 may be disposed within head 12 or within handle 14. Channels 76 may extend from reservoir 74 to an interior portion of head 12 or to outer surface 40 of head 12. In one embodiment, reservoir 74 may be disposed within handle 14 and a tab 78 may be depressed for releasing solution 72 through channels 76 to head 12.

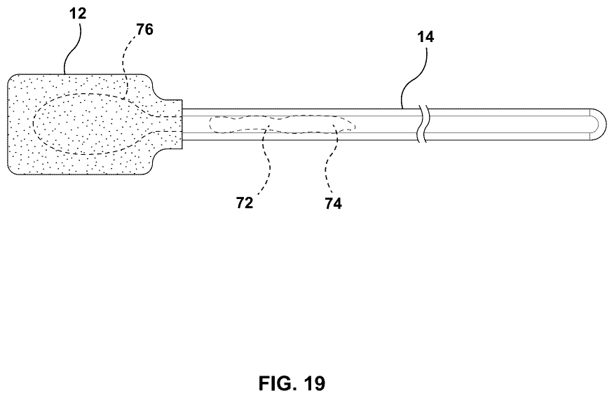

In another embodiment, as shown in FIG. 19, handle 14 may be hollow to define reservoir 74 and handle 14 may be formed of a pliable material (such as a pliable plastic material) such that handle 14 may be squeezed by user to cause solution 72 to be distributed through channels 76 to head 12.

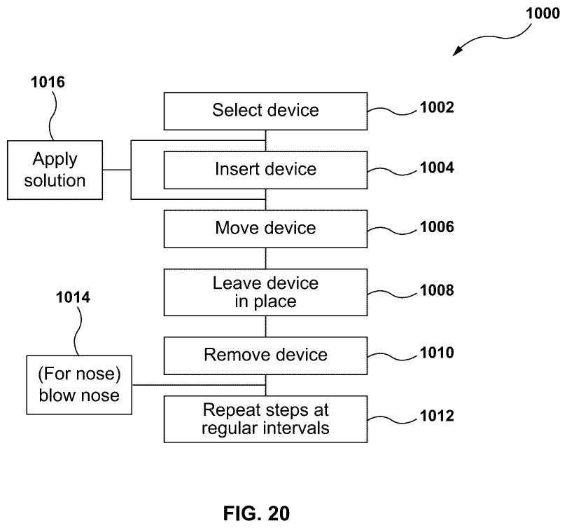

Referring to FIG. 20, a preferred method of use for device 10 is shown at 1000. Method 1000 includes step 1002 of selecting device 10 (this may be a disposable single use device 10 or a device 10 where handle 14 is reusable with disposable single use heads 12). Method further includes step 1004 where head 12 of device 10 is gently inserted a desired distance into body orifice 16. Method then proceeds to step 1006 where head of device 10 is moved within body orifice (in a preferred embodiment, movement back and forth five times is preferred) to enable optimum collection of substances 18. It will be understood that head 12 of device 10 may not collect all substances located within body orifice 16 during each use but should collect at least some substances, and preferably a substantial amount of substances, to reduce the amount of substances within body orifice 16. Method then proceeds to step 1008 where head 12 of device 10 is left in place within body orifice 16 for a desired period of time to allow substances 18 to further collect (in a preferred embodiment, the desired period of time is between 5-30 seconds and more preferably between 10-20 seconds). Method then proceeds to step 1010 where head 12 of device 10 with collected substances 18 is removed from body orifice 16 and device is disposed into a suitable waste receptacle. Method 1000 then moves to step 1012 where steps 1002 to 1010 are repeated at regular intervals (preferably multiple times per week as part of a regular hygiene practice, more preferably at least once per day, more preferably at least once per day and/or after exposure to potentially harmful substances such as viruses while user is attending work, clubs, restaurants, school, gym or in close contact with others that may have been exposed to such harmful substances).

Where body orifice 16 is a nose, a user may select a new device 10 having an unused head 12 for each nostril (or, in instances where the head alone may be replaced, the user may dispose of existing head and select an unused head 12 for the other nostril). Alternatively, a user may reuse the same device 10 for both nostrils and repeat method steps 1004-1008 before disposal step 1010. Alternatively, in the case of a device having a head 12 at each end of device 10, device 10 may be flipped so that the remaining unused head 12 is used in the other nostril. Following disposal step 1010, where body orifice 16 is a nose, method 1000 includes step 1014 where user blows their nose with a tissue to allow any remaining amounts of substances 18 to be removed from the nose. Tissue may then be discarded in a similar manner to disposal step 1010.

A variation of method 1000 includes additional step 1016 where user applies solution 72, preferably a saline solution, to head 12 either prior to step 1004 of gently inserting head 12 into orifice 16 or during the time when head 12 is disposed in body orifice 16. Solution 72 may be applied to head 12 by squirting an amount of solution 72 onto head 12 or by injecting an amount of solution 72 into one or more pores of head or by dipping head 12 into a container with solution 72. Alternatively, device 10 may be provided with dispensing system 70 as described earlier for dispensing a desired volume of solution 72 into or onto head 12 prior to or during the time when head 12 is disposed in body orifice 16.

It is believed that use of device 10 on a regular basis and/or after exposure to harmful substances will reduce the risk of harmful substances such as viruses moving from body orifice 16 to the remainder of the living body. For example, where body orifice 16 is a nose, it is believed that use of device 10 with both nostrils of the nose on a regular basis and/or after exposure to harmful substances will reduce the risk of harmful substances such as viruses moving from the nose and entering the body.

While the above description provides examples of one or more processes or apparatuses, it will be appreciated that other processes or apparatuses may be within the scope of the accompanying claims.

I/We claim:

1. A hygiene device for use with a body orifice of a living being, the hygiene device comprising:
a) a head adapted for use in the body orifice, said head being formed of one or more absorbent materials that are adapted to collect substances from the body orifice; and
b) a handle connected to said head to enable said head to be inserted into the body orifice to collect substances and to be subsequently removed from the body orifice to remove the collected substances.

2. The hygiene device as claimed in claim 1 wherein said head is formed from an open cell polyurethane foam material.

3. The hygiene device as claimed in claim 1 further comprising a flange that is adapted to limit the extent to which said head may be inserted into the body orifice.

4. The hygiene device as claimed in claim 1 wherein said device includes a plurality of heads.

5. The hygiene device of claim 1 wherein said device includes two of said heads with each of said two heads being disposed at an opposing end of said handle.

6. The hygiene device of claim 1 wherein said head is releasably attached to said handle.

7. The hygiene device of claim 1 wherein said handle includes a latch for releasably attaching said head to said handle.

8. The hygiene device of claim 1 wherein said head has a volumetric shape that generally corresponds to the volumetric shape of said orifice.

9. The hygiene device of claim 1 wherein said head has a volume that is adapted to fill at least 25% of the volume of said orifice.

10. The hygiene device of claim 1 wherein said head has an asymmetric volumetric shape.

11. The hygiene device as claimed in claim 1 wherein said orifice comprises the anterior nares.

12. The hygiene device as claimed in claim 11 wherein said head has a first face having a generally curved or convex shape adapted to match the generally curved or concave shape of the alae of the anterior nares and said head has a second face, opposing said first face, having a generally flat shape adapted to match the generally flat shape of the septum of the anterior nares.

13. The hygiene device of claim 1 wherein said device includes a dispensing system for dispensing a desired solution to said head.

14. The hygiene device of claim 13 wherein said dispensing system includes a reservoir adapted for containing said solution and one or more channels for transferring said solution from said reservoir to said head.

15. A method of using a device in a body orifice of a living body to collect and remove substances from the body orifice where such device includes a head formed of one or more absorbent materials that are adapted to collect substances from the body orifice and a handle connected to the head for inserting and removing the head from the body orifice, said method including the steps of:
a) selecting a device;
b) inserting the head of the device into the body orifice to collect substances from the body orifice;
c) removing the head of device from the body orifice with the collected substances.

16. The method as claimed in claim 15 further comprising, after the inserting step, the step of moving the head of the device within body orifice to further collect substances from the body orifice.

17. The method as claimed in claim 15 further comprising, after the inserting step, the step of leaving the head of the device within the body orifice for a desired period of time to further collect substances from the body orifice.

18. The method as claimed in claim 15 further comprising the step of applying a desired solution to said head either prior to or during the time when said head is inserted into or disposed within said orifice.

19. The method as claimed in claim 15 further comprising, after the removing step, in instances where orifice comprises the anterior nares, blowing the nose.

20. Use of a device in a body orifice of a living body to collect and remove substances where such device includes a head adapted for use in the body orifice, said head being formed of one or more absorbent materials that are adapted to collect substances from the body orifice; and a handle connected to said head for inserting and removing said head with collected substances from the body orifice.
